(19) United States
(12) Patent Application Publication
(10) Pub. No.: US 2025/0213598 A1
(43) Pub. Date: Jul. 3, 2025

(54) COMPOUNDS AS ELASTASE INHIBITOR AND SOLID FORM THEREOF

(71) Applicant: SHANGHAI ARK BIOPHARMACEUTICAL CO. LTD., Shanghai (CN)

(72) Inventors: Weikai GU, Shanghai (CN); Mixia WANG, Shanghai (CN); Feifei GE, Shanghai (CN); Qiwu ZHAO, Shanghai (CN); Haiqing YUAN, Shanghai (CN)

(21) Appl. No.: 19/002,107

(22) Filed: Dec. 26, 2024

(30) Foreign Application Priority Data
Dec. 29, 2023 (CN) 202311866446.8

(51) Int. Cl. A61K 31/69; A61P 11/00; C07F 5/04

(52) U.S. Cl. CPC A61K 31/69; A61P 11/00; C07F 5/04

(57) ABSTRACT

The present disclosure relates to the compound of an elastase inhibitor having the structure of Formula (I), the crystal form of the said inhibitor and its preparation method, the amorphous form of the said inhibitor and its preparation method and applications of these. The preparation method in present disclosure has advantages in simplicity, good repeatability, strong drug induction ability, high in-vivo exposure, long pharmacokinetic elimination half-life and high safety. The crystal form or amorphous form of present compound has beneficial effects such as high solubility and good stability. The preparation methods for the crystal form or amorphous form of the present compound are simple, easy to control and have a good reproducibility in the crystallization process.

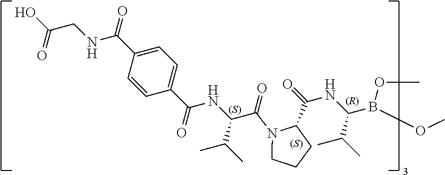

### TECHNICAL FIELD

The present disclosure belongs to the field of medicine, and particularly relates to a compound as an elastase inhibitor and its applications, the crystal forms of the compound and their preparation methods and applications, and the amorphous form of the compound and its preparation method and application.

### BACKGROUND

Human neutrophil elastase (HNE), also known as human leukocyte elastase (HLE), is a 32 kDa serine protease and a member of the neutrophil serine protease (NSPs) family. Currently, HNE is considered to be a multifunctional enzyme with various proteolytic activities. It can participate in pathogen killing, inflammation regulation, and maintenance of tissue homeostasis, thereby becoming the important protective barrier when the body is threatened. However, excessive NHE is highly destructive to the human body because the over-released HNE will cause direct tissue damage by hydrolyzing its specific substrates, and lead to symptoms of acute infections, acute hemolysis and blood loss, tissue damage or necrosis, acute poisoning and malignant tumors by participating inflammatory reactions in various organs. As a result, the liver specifically synthesizes and secretes a natural inhibitor (α-1 antitrypsin) to maintain the balance of the elastase activity in the human body.

Elastin is one of the main components of the extracellular matrix protein, and other components include fibronectin, laminin, proteoglycan, type III and type IV collagen. The functions of the elastase include acting against bacterial invasions by degrading bacterial structural proteins. In short, the human elastase can degrade damaged tissues or invading bacteria and thus plays an indispensable role in maintaining the body's homeostasis.

The imbalance of elastase content is a basic characteristic of serious diseases, especially those related to the cardiopulmonary system, such as chronic obstructive pulmonary disease (COPD), bronchiectasis (BE), cystic fibrosis (CF), acute lung injury (ALI), acute respiratory distress syndrome (ARDS), pulmonary artery hypertension (PAH), and idiopathic pulmonary fibrosis (IPF). When inflammation occurs, unlike under normal physiological conditions, activated neutrophils release excessive HNE, upregulate cell adhesion factors to promote the adhesion of neutrophils to endothelial cells, and release pro-inflammatory molecules to induce cytokine expression. These substances can in turn activate neutrophils to release more HNE. Among them, chronic obstructive pulmonary disease is a disease with high incidence. Currently, there are approximately 100 million patients with chronic obstructive pulmonary disease in China, and this number is still increasing due to factors such as population aging and air pollution. There is no effective treatment for this disease so far, and the currently used bronchodilators (LABA/LAMA) can only relieve symptoms without stopping the progression of the disease. These patients will still continuously suffer from pulmonary infections, and after each episode of inflammation, the lung function impairment will become more severe.

At present, there are extensive researches on using elastase inhibitors to treat this type of inflammatory disease abroad, and the first marketed drug, sivelestat sodium hydrate, is the first successfully developed drug that treats such diseases by elastase inhibition. In addition, several inhibitors have entered or are in clinical trials to test their therapeutic effects on diseases such as chronic obstructive pulmonary disease, cystic fibrosis, or α-1 antitrypsin deficiency.

However, the existing compounds have a short in vivo exposure time and are rapidly eliminated, which may weaken their efficacy. Therefore, it is necessary to develop a class of elastase inhibitors with extended in vivo exposure time and improved exposure amount.

### SUMMARY

### Problems to be Solved by Present Invention

Based on the fact that the existence forms and quantities of polymorphic compounds are unpredictable, the technical problem to be solved by present disclosure is to overcome the deficiencies in the prior art and provide a compound of elastase inhibitor with the structure of Formula (I), and the preparation methods and applications of its crystal form A and amorphous form. The elastase inhibitor compound and its crystal form A and amorphous form in present disclosure have stable physical and chemical properties and good druggability. The preparation methods in present disclosure are simple and easy to repeat.

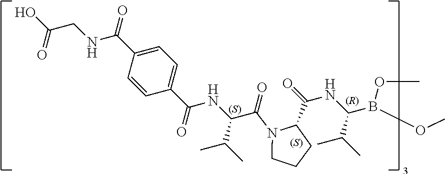

### Solutions to the Problem

Provided in present disclosure is a compound of Formula (I) or a pharmaceutically acceptable salt, ester, isomer, solvate, prodrug or isotopically labeled derivative thereof,

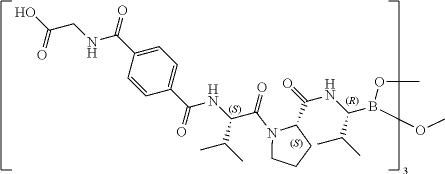

Provided in present disclosure is the crystal form A of the compound of Formula (I), having

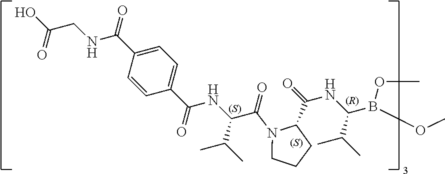

An X-ray powder diffraction pattern comprising, in terms of 2θ angle, peaks at 8.48, 9.18, 13.09, 17.08 and 18.47; preferably, peaks at 8.48, 9.18, 10.32, 11.46, 13.09, 16.14, 16.73, 17.08, 18.47, 21.09 and 21.62; more preferably, peaks at 8.48, 9.18, 10.32, 11.46, 11.70, 13.09, 13.52, 15.76, 16.14, 16.73, 17.08, 18.47, 21.09, 21.62, 22.87 and 25.99; most preferably, the X-ray powder diffraction pattern substantially as shown in FIG. 2.

Provided in present disclosure is an amorphous form of the compound of Formula (I), having an X-ray powder diffraction pattern substantially as shown in FIG. 4, in terms of 2θ angle.

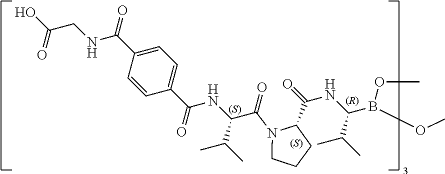

### Effects of the Disclosure

1. The preparation method of the elastase inhibitor compound shown in Formula (I) of present disclosure is simple and has good repeatability, and has the advantages of strong drug induction ability, high in vivo exposure, extended pharmacokinetic elimination half-life, and high safety.
2. The crystal form A and amorphous form of the elastase inhibitor compound shown in Formula (I) of present disclosure have advantages in physical and chemical properties, formulation processing performance, and bioavailability, such as the at least one advantage selected from melting point, solubility, hygroscopicity, purification effect, stability, adhesiveness, compressibility, fluidity, in vitro and in vivo dissolution, and bioavailability. The crystal form or amorphous form of the elastase inhibitor compound shown in Formula (I) of present disclosure has good physical and chemical stability. The preparation method disclosed in present disclosure is able to achieve a high yield of the crystal form or amorphous form using the same starting material, and the achieved crystal form or amorphous form has obvious advantages in solubility, hygroscopicity, stability, mechanical stability, fluidity, compressibility, and adhesiveness, thereby providing a new and better choice for the drug development of diseases mediated by elastase and having very important significance.

### BRIEF DESCRIPTION OF THE DRAWINGS

FIG. 1 shows the high-resolution mass spectrum of the compound shown in Formula (I).

FIG. 2 shows the XRPD pattern for the crystal form A of the compound shown in Formula (I).

FIG. 3 shows DSC & TGA diagrams of the crystal form A of the compound shown in Formula (I).

FIG. 4 shows the XRPD pattern of the amorphous solid of the compound shown in Formula (I).

FIG. 5 shows DSC & TGA diagrams of the amorphous solid of the compound shown in Formula (I).

FIG. 6 shows the XRPD diagrams of crystal form A of the compound shown in Formula (I) after dry milling and wet milling at room temperature.

### DETAILED DESCRIPTION

To make the technical solutions and beneficial effects of present disclosure more obvious and easier to understand, the following description is explained in detail by enumerating specific embodiments. The drawings are not necessarily drawn to scale, and local features can be enlarged or reduced to show the details of local features more clearly. Unless otherwise defined, the technical and scientific terms used in this text have the same meanings as those in the technical field to which present disclosure belongs.

Provided in present disclosure is a compound of formula I, or a pharmaceutically acceptable salt, ester, isomer, solvate, prodrug or isotopically labeled derivative thereof

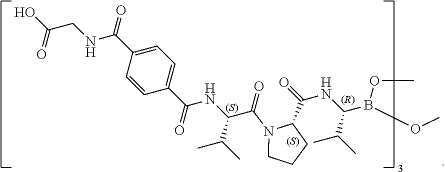

Provided in present disclosure a crystal form A of the compound of Formula (I), having

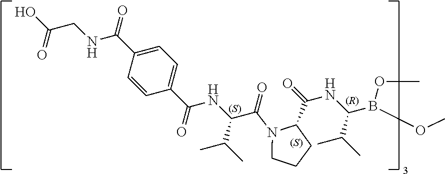

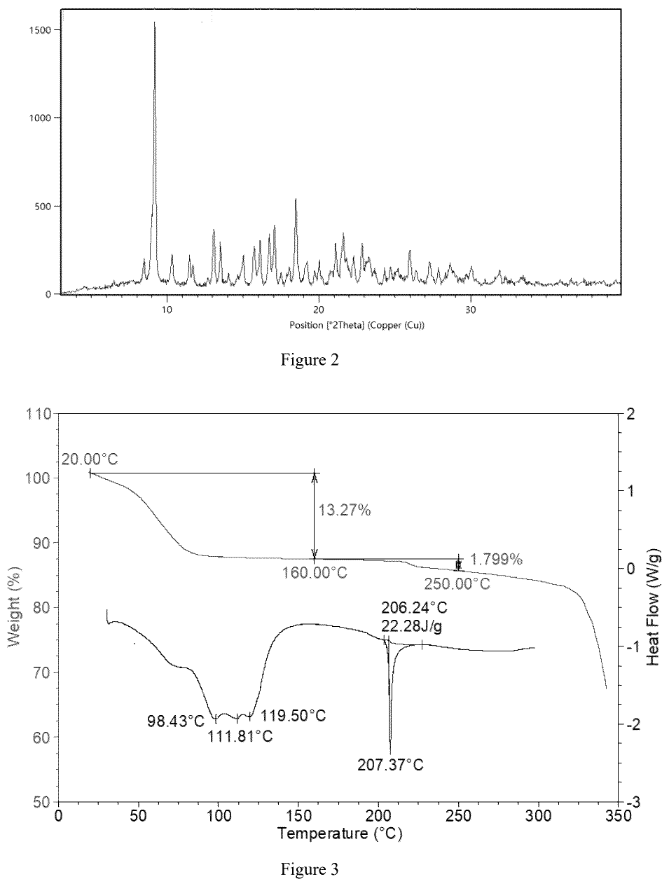

an X-ray powder diffraction pattern comprising, in terms of 2θ angle, peaks at 8.48, 9.18, 13.09, 17.08 and 18.47; preferably, peaks at 8.48, 9.18, 10.32, 11.46, 13.09, 16.14, 16.73, 17.08, 18.47, 21.09 and 21.62; more preferably, peaks at 8.48, 9.18, 10.32, 11.46, 11.70, 13.09, 13.52, 15.76, 16.14, 16.73, 17.08, 18.47, 21.09, 21.62, 22.87 and 25.99; most preferably, the X-ray powder diffraction pattern substantially as shown in FIG. 2.

Further provided in present disclosure is a preparation method of the crystal form A of the compound shown in Formula (I), including the steps of using the amorphous form of the compound of Formula (I) as a raw material and preparing the crystal form A by a method selected from gas-solid diffusion, gas-liquid diffusion, slow evaporation, suspension stirring at room temperature to 50° C., temperature cycle stirring, slow-cooling and polymer induction.

Further provided in present disclosure is a preparation method of the crystal form A of the compound shown in Formula (I), including the steps of mixing the compound of Formula (I) with an aqueous solution of solvent 1, and then stirring and filtering.

In some embodiments, the solvent 1 is selected from nitrile solvents.

In some embodiments, the solvent 1 is selected from the group consisting of acetonitrile, trimethylacetonitrile, propionitrile, valeronitrile and combination thereof.

In some embodiments, the solvent 1 is acetonitrile.

In some embodiments, the preparation method of present disclosure further includes the step of such as centrifugation, washing, or drying.

In some embodiments, the volume ratio of solvent 1 to water is (5-10):1.

In some embodiments, the volume ratio of solvent 1 to water is 8.8:1.2.

Further provided in present disclosure is an amorphous form of the compound of Formula (I),

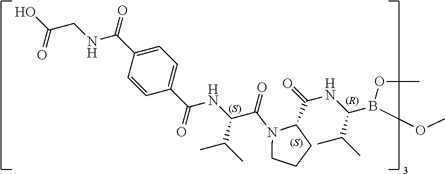

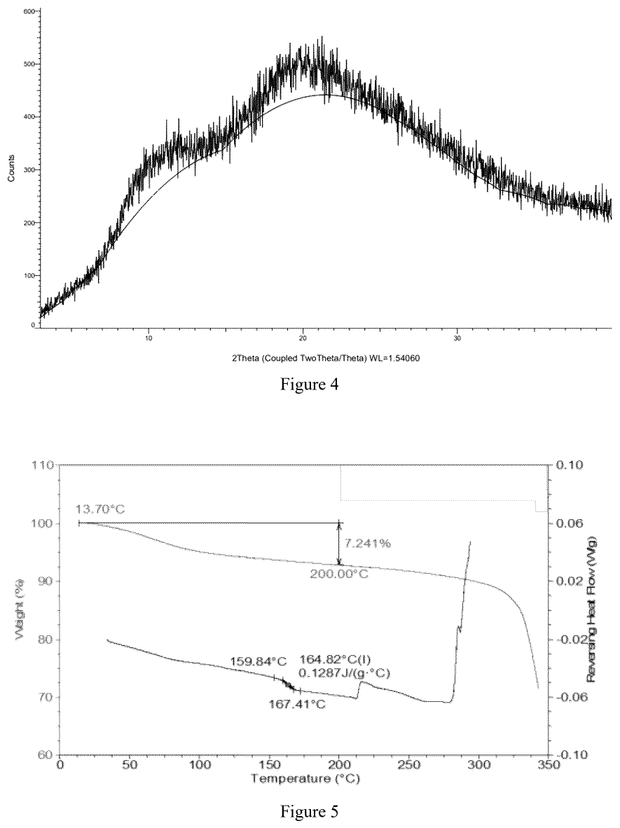

having an X-ray powder diffraction pattern substantially as shown in FIG. 4, in terms of 2θ angle.

Further provided in present disclosure is a preparation method of the amorphous form of the compound shown in Formula (I), including the steps of adding solvent 3 to the solution 2 of the compound shown in Formula (I), stirring, and filtering.

In some embodiments, the preparation method of the amorphous form in present disclosure further includes one or more steps selected from the group consisting of centrifugation, washing and drying.

In some embodiments, the solvent of solution 2 is one or more selected from the group consisting of 1,4-dioxane, tetrahydrofuran, 2-methyltetrahydrofuran, ethylene glycol dimethyl ether, dichloromethane, dimethyl sulfoxide, acetone, acetonitrile, and water.

In some embodiments, the solvent of solution 2 is the mixture of acetonitrile and water.

In some embodiments, the volume ratio of acetonitrile to water is (4-9):1.

In some embodiments, the volume ratio of acetonitrile to water is 4:1.

In some embodiments, the solvent 3 is selected from the group consisting of methyl tert-butyl ether, acetonitrile, and isopropyl acetate.

In some embodiments, the solvent 3 is acetonitrile.

In some embodiments, the volume ratio of solution 2 to solvent 3 is 1:(5-15).

In some embodiments, the volume ratio of solution 2 to solvent 3 is 1:(5-10).

Further provided in present disclosure is a pharmaceutical composition prepared from the aforementioned compound of Formula (I) or a pharmaceutically acceptable salt, ester, isomer, solvate, prodrug or isotopically labeled derivative thereof, or the aforementioned crystal form A of the compound of Formula (I), or the aforementioned amorphous form of the compound of Formula (I).

Further provided in present disclosure is a pharmaceutical composition including the aforementioned compound of Formula (I) or a pharmaceutically acceptable salt, ester, isomer, solvate, prodrug or isotopically labeled derivative thereof, or the aforementioned crystal form A of the aforementioned compound of Formula (I), or the aforementioned amorphous form of the compound of Formula (I), and any pharmaceutically acceptable excipient.

“Pharmaceutically acceptable excipient”, as used herein, refers to pharmaceutically acceptable materials, mixtures, or solvents related to the consistency of dosage forms or the pharmaceutical compositions. Suitable pharmaceutically acceptable excipients will vary depending on the selected dosage form. In addition, pharmaceutically acceptable excipients can be selected according to their specific functions in the composition.

Further provided in present disclosure is a preparation method of a pharmaceutical composition, including mixing the aforementioned compound of Formula (I) or a pharmaceutically acceptable salt, ester, isomer, solvate, prodrug or isotopically labeled derivative thereof, or the aforementioned crystal form A of the compound of Formula (I), or the aforementioned amorphous form of the compound of Formula (I), with pharmaceutically acceptable excipients.

Further provided in present disclosure the aforementioned compound of Formula (I) or a pharmaceutically acceptable salt, ester, isomer, solvate, prodrug or isotopically labeled derivative thereof, or the aforementioned amorphous form of the compound of Formula (I), or the aforementioned pharmaceutical compositions, or the pharmaceutical composition prepared by the aforementioned method, for use in a preparation of medicaments for treating and/or preventing diseases mediated by an elastase.

In some embodiments, the diseases mediated by elastase is selected from chronic obstructive pulmonary disease, bronchiectasis, cystic fibrosis, acute lung injury, chronic bronchitis, acute respiratory distress syndrome, pulmonary artery hypertension, idiopathic pulmonary fibrosis and alpha-1 antitrypsin deficiency.

In some embodiments, the diseases mediated by elastase is the chronic obstructive pulmonary disease (COPD).

The “2θ or 2θ angle”, as used herein, refers to the diffraction angle, where θ is the Bragg angle, and the unit is ° or degree. The error range of each characteristic peak 2θ is ±0.2 (including the case where numbers with more than 1 decimal place are rounded off), and can be −0.20, −0.19, −0.18, −0.17, −0.16, −0.15, −0.14, −0.13, −0.12, −0.11, −0.10, −0.09, −0.08, −0.07, −0.06, −0.05, −0.04, −0.03, −0.02, −0.01, 0.00, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20.

The precipitation methods in present disclosure include but are not limited to stirring, cooling, evaporation, slurrying, and precipitation.

“Slurrying”, as used herein, is a common term in the field of drug preparation and usually refers to the mechanization or fluidization of raw solid drug materials (also named API) to disperse or suspend the solid drug in solvent.

In some embodiments, the slurrying time is from 5 to 30 hours.

The “differential scanning calorimetry or DSC”, as used herein, refers to measuring the temperature difference and heat flow difference between a sample and a reference during the heating or constant temperature process of the sample to characterize all physical and chemical changes related to the heat effect and thus obtain the phase change information of the sample.

The drying temperature in present disclosure is generally 25° C.-100° C., preferably 40° C.-70° C., and the drying procedure can be performed at a normal pressure or under reduced pressure.

The following embodiments describe the method of present disclosure for illustration. It should be understood that these embodiments are used to illustrate the basic principles, main features, and advantages of present invention that is not limited by the scope of the following embodiments. The experimental conditions adopted in the embodiments can be further adjusted according to specific requirements, and the unstated experimental conditions are usually those in conventional experiments.

The reagents used herein can be obtained through commercial ways.

The test conditions of the instruments used in the experiments of present disclosure are as follows:

### 1. X-Ray Powder Diffraction (XRPD)

The crystal form of the sample is analyzed by Bruker D2 X-ray diffractometer at a 2θ scanning angle of 3°-40°, a scanning step sizer of 0.02°, and a scanning frequency of 0.15 s/step. The voltage and current of the X-ray tube are 30 kV and 10 mA, respectively. When preparing the sample, an appropriate amount of sample is placed on the sample tray and flattened with tools such as glass slides to ensure a smooth surface.
2. Thermogravimetric Analysis (TGA) and Differential Scanning calorimetry (DSC)

Mettler-Toledo TGA/DSC3+ thermogravimetric analyzer and differential scanning calorimeter are combined to analyze the sample. The sample is placed in an aluminum pan with the tare removed, and automatically weighed by the system. Then, the sample is heated from 30° C. to 450° C. at a rate of 10° C./min under the protection of 25 mL/min nitrogen.
3. High-Performance Liquid Chromatography (HPLC)

The solubility and stability are analyzed using an Agilent 1260 infinityII Binary Pump.
4. Nuclear Magnetic Resonance Hydrogen Spectrum (¹H NMR Spectrum)

¹H NMR Spectrum is measured using a Bruker instrument (400 MHz) with the chemical shift expressed in ppm, and D2O used as the internal standard (0.00 ppm). The ¹H NMR is shown as follow: s=singlet, d=doublet, t=triplet, q=quartet, m=multiplet, br=broad peak, dd=doublet of doublets, dt=doublet of triplets. If the coupling constant is provided, its unit is Hz.
5. High-Resolution Mass Spectrometry (MALDI-TOF MS)

High-Resolution mass spectrometry is measured using a MALDI-8030 MALDI-TOF MS, and the ionization method is negative ion.
6. Elemental Analysis

Elemental analysis is analyzed using a Vario Micro Cube automatic elemental analyzer and a Leeman Prodigy inductively coupled plasma emission spectrometer. C, H, and N are detected using the automatic elemental analyzer. B is tested using ICP-OES. O is calculated according to the results of C, H, N, and B based on mass conservation.

### Example 1 Preparation of the Compound of Formula (I)

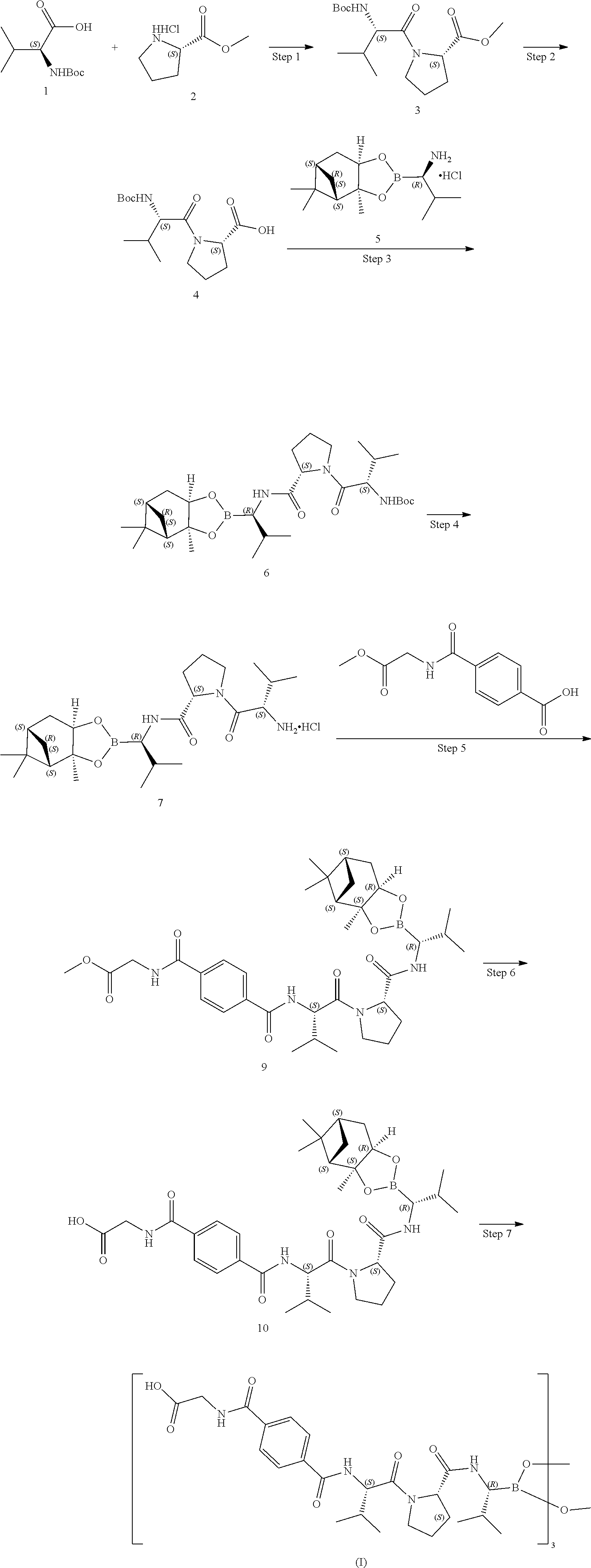

Step 1: Dichloromethane (1320.0 g) was added to a dry three-necked flask and stirred under nitrogen protection following the addition of compound 2 (84.0 g). Then, the temperature was controlled to 20° C., and DIPEA (2.0 eq, 119.0 g) was added dropwise to the three-necked flask under nitrogen protection. After the addition, the mixture was stirred for 1-2 h and marked as system R1. Dichloromethane (1320.0 g) was added to a dry jacketed flask, stirred under nitrogen protection and added with 100.0 g of compound 1. The temperature was controlled to 20° C., and CDI (1.05 eq, 78.4 g) was added dropwise in batches following with stirring at 25° C. for 2-4 h. Then, the solution of system R1 was added to the jacketed flask with the temperature kept at 10-30° C. and stirred at 25° C. for 5-7 h. After the reaction was completed, the reaction mixture was added with water (200.0 g) under stirring at a temperature below 25° C. and placed for stratification. Then, the resulting organic phase was transferred to the reactor under stirring, was added with 1 mol/L hydrochloric acid aqueous solution (1000.0 g) under stirring at a temperature of 20° C. and placed for stratification. Then, the resulting organic phase was transferred to the reactor under stirring, added with 10% sodium bicarbonate aqueous solution (1000.0 g) at a temperature of 20° C. under stirring and placed for stratification. The resulting organic phase was transferred to the reactor under stirring, added with water (500.0 g) at a temperature of 25° C. under stirring, and placed for stratification. The resulting organic phase was concentrated under reduced pressure to 100-200 mL to give a colorless to pale yellow solution. Tetrahydrofuran (5 V, 446.0 g) was added to the solution that was then concentrated under reduced pressure to 100-200 mL. Then, tetrahydrofuran (89.0 g) was added again to the solution that was stirred to give a colorless to pale yellow solution. The solution contains 120.0 g of compound 3 with a yield of 79.4% and a purity of ≥95% by calculation.

The characterization data of compound 3:

¹H NMR (400 MHZ, DMSO-d6) δ 6.88-6.37 (m, 1H), 4.32 (dd, J=8.5, 5.4 Hz, 1H), 403 (t, J=8.3 Hz, 1H), 3.77 (d, J=9.9 Hz, 1H), 3.61 (s, 4H), 2.28-2.08 (m, 1H), 2.04-1.75 (m, 4H), 1.37 (s, 9H), 0.89 (dd, J=19.2, 6.7 Hz, 6H).

MS [M+H]⁺=329

Step 2: Water (131.0 g) and lithium hydroxide monohydrate (25.5 g) were added to a beaker and stirred to dissolve for later use. The jacketed flask containing tetrahydrofuran (267.0 g) was added with the solution (1.00 eq, 100.0 g) of compound 3 in tetrahydrofuran (450 mL) under stirring and nitrogen protection, to which the lithium hydroxide aqueous solution was added dropwise under at 0-5° C. and kept stirring for 2-3 h. After the reaction was completed, the reaction solution was added with 2% sodium hydroxide aqueous solution (265 g) at a temperature of 0-5° C., and then with methyl tert-butyl ether (222.0 g) under stirring. The resulting solution was placed for stratification and given the aqueous phase (W1) for the next step and the organic phase (O1) for temporary storage. The aqueous phase (W1) was added with Methyl tert-butyl ether (222.0 g) at 20° C., stirred and placed for stratification to give the aqueous phase (W2) for temporary storage. The organic phase (O1) was transferred to the extraction kettle, added with water (200 g) under stirring and placed for stratification to give the aqueous phase (W3) for temporary storage. The combined aqueous phase (W2 and W3) was transferred to the reactor under stirring, added with methyl tert-butyl ether (148.0 g) at 20° C. under stirring, and placed for stratification. The resulting aqueous phase was added to the reaction flask following with addition of dichloromethane (585.2 g) under stirring, cooled down to 0-5° C., added with 2 mol/L hydrochloric acid aqueous solution (355.0 g) dropwise to adjust the pH to 3-4 under stirring, and placed for stratification. The resulting aqueous phase was added with dichloromethane (264.0 g) under stirring, placed for stratification to give the organic phase that was then concentrated to about 100 mL under reduced pressure to obtain a colorless to pale yellow solution. This solution was added with n-heptane (680.0 g) dropwise, stirred for 2 h, cooled down to 0-10° C., stirred for 1-2 h, and filtered. The filter cake was washed with n-heptane (68.0 g) and dried to give a white to off-white solid (compound 4, 83.0 g) with a yield of 86.7% and a purity of ≥97.0%.

The characterization data of compound 4:

¹H NMR (400 MHZ, DMSO-d6) δ 12.33 (s, 1H), 6.79 (d, J=8.6 Hz, 1H), 4.24 (dd, J=8.6, 5.0 Hz, 1H), 4.01 (t, J=8.3 Hz, 1H), 3.82-3.69 (m, 1H), 3.56 (dt, J=9.7, 6.6 Hz, 1H), 2.16 (ddt, J=12.1, 8.7, 7.1 Hz, 1H), 1.92 (d, J=6.2 Hz, 3H), 1.82 (dq, J=12.2, 6.4, 5.8 Hz, 1H), 1.37 (s, 10H), 0.89 (dd, J=20.8, 6.7 Hz, 6H).

MS [M+Na]=337

Step 3: Under nitrogen protection, compound 4 (1.00 eq, 2.10 g), compound 5 (1.00 eq, 1.91 g) and THF (21 g) were successively added, and cooled down to −50 to −60° C. following with addition of DIPEA (4.3 g) dropwise, stirred for 10-30 min at this temperature, and added with 50% T4P ethyl acetate solution (9.5 g) dropwise. After the addition, the solution was allowed to heat up naturally to a temperature ranging from −20° C. to 0° C. for the reaction. The reaction solution was transferred to the reactor after the reaction is completed. Potassium carbonate (2.1 g) was added to purified water (18 g) and dissolved under stirring for later use. The prepared 10% potassium carbonate aqueous solution was added to the reactor dropwise at 5° C. Then, reaction solution was added with MTBE (15 g) stirred and placed for stratification to give the organic phase 3-A for temporary storage. MTBE (7.5 g) was again added to the resulting aqueous phase which was stirred and placed for stratification to give the organic phase 3-B for temporary storage. The organic phases (3-A and 3-B) were combined to give the organic phase 3-C. Concentrated hydrochloric acid (1.6 g) was added to purified water (18 g) and stirred well for later use. The organic phase 3-C was added with the prepared hydrochloric acid aqueous solution at 0° C., stirred and placed for stratification to give the organic phase 3-D. The organic phase 3-D was added with purified water (10 g) at 0° C. for extraction twice. The resulting organic phase was taken and concentrated until there was no obvious distillate left. THF (10 g) was added and dissolved in this solution that was concentrated until there was no obvious distillate left. Then, repeat this operation. THF (8.0 g) was added to the resulting solution and stirred to give THE solution of compound 6 that can be directly used in the next step. The THE solution of compound 6 was tested by HPLC and calculated to obtain 3.14 g of compound 6 at a purity of 98% and a yield of 90%.

The characterization data of compound 6:

¹H NMR (400 MHZ, DMSO-d6) δ 8.86 (t, J=3.3 Hz, 1H), 6.84 (d, J=8.5 Hz, 1H), 4.49 (dd, J=8.6, 4.3 Hz, 1H), 4.09-3.95 (m, 2H), 3.75-3.55 (m, 2H), 2.25-1.58 (m, 13H), 1.37 (s, 9H), 1.21 (d, J=6.9 Hz, 6H), 0.92-0.80 (m, 15H).

MS [M+H]⁺=548

Step 4: Under nitrogen protection, 24.0 g of the tetrahydrofuran solution of compound 6 (content: 25%) was heated to 30-40° C., stirred until it becomes clear, and added with HCl in 1,4-dioxane solution (4 mol/L, 18 g) dropwise, and stirred for 12-16 h at 35° C. After the reaction was completed, the reaction solution was concentrated under reduced pressure until there was no obvious distillate left. The concentrated residue was added with tetrahydrofuran (20 g), stirred to clear, concentrated under reduced pressure until there was no obvious distillate left. Repeat this operation twice. The concentrated residue was added with tetrahydrofuran (18 g), stirred to clear, aged for 10-12 h at 50° C., added with n-heptane slowly, cooled down to 5-15° C., and filtered. The filter cake was washed with heptane, and dried to give a white solid (5.1 g) having a purity of 99.3% and a yield of 88%.

The characterization data of compound 7:

¹H NMR (400 MHz, DMSO-d6) δ 8.80 (t, J=3.9 Hz, 1H), 8.16 (d, J=5.2 Hz, 3H), 4.53 (dd, J=8.3, 5.4 Hz, 1H), 4.10 (dd, J=8.7, 2.2 Hz, 1H), 3.96 (t, J=5.4 Hz, 1H), 3.74 (dt, J=9.9, 6.4 Hz, 1H), 3.57-3.52 (m, 1H), 2.41 (dd, J=5.7, 3.5 Hz, 1H), 2.25-2.10 (m, 3H), 2.06-1.95 (m, 2H), 1.89-1.71 (m, 5H), 1.65 (dt, J=13.9, 2.8 Hz, 1H), 1.35 (d, J=10.1 Hz, 1H), 1.22 (d, J=2.3 Hz, 6H), 1.01 (d, J=6.9 Hz, 3H), 0.95 (d, J=6.8 Hz, 3H), 0.89 (dd, J=13.1, 6.8 Hz, 6H), 0.81 (s, 3H).

MS [M+H]⁺=448

Step 5: Under nitrogen protection, compound 7 (5 g), compound 8 (2.55 g) and tetrahydrofuran (45 g) were successively added, cooled down to −30 to −40° C., added with DIPEA (6.7 g) dropwise, stirred for 20-40 min, added with 50% T4P ethyl acetate solution (14.9 g) dropwise and raised to −10 to 10° C. for the reaction. After the reaction was completed, 10% potassium carbonate aqueous solution (50 g) was added to the reaction solution dropwise. Then, methyl tert-butyl ether (27.4 g) was added and stirred. The reaction solution was placed for stratification to give the aqueous phase and the organic phase 5-A that is temporarily stored. Methyl tert-butyl ether (13.7 g) was added to the aqueous phase that was stirred, placed for stratification to give the aqueous phase and the organic phase 5-B for temporary storage. The organic phase 5-C obtained from combining organic phases 5-A and 5-B was added with

1M hydrochloric acid aqueous solution (50 g), stirred, placed for stratification to give the aqueous phase and the organic phase 5-D that is temporarily stored. The organic phase 5-D was added with purified water (25 g) for extraction to give the bottom aqueous phase and the organic phase that was temporarily stored. Repeat this operation and combine the organic phases of the two extractions to give the organic phase 5-E. The organic phase 5-E was concentrated until there was no obvious distillate left. After the addition of tetrahydrofuran (20 g), the concentrated residue was concentrated under reduced pressure until there was no obvious distillate left in twice. Tetrahydrofuran (20 g) was added to the concentrated residue to give the THF solution of compound 9 that can be directly used in the next step. The THF solution of compound 9 was tested using HPLC and calculated to obtain 6.18 g of compound 9 at a purity of 96.8% and a yield of 90%.

The characterization data of compound 9:

¹H NMR (400 MHZ, DMSO-d6) δ 9.09 (t, J=5.9 Hz, 1H), 8.87 (t, J=3.5 Hz, 1H), 8.69 (d, J=8.1 Hz, 1H), 8.06-7.89 (m, 4H), 4.50 (d, J=4.0 Hz, 1H), 4.05 (dd, J=12.1, 6.1 Hz, 3H), 3.89 (dt, J=9.8, 7.1 Hz, 1H), 3.66 (d, J=8.9 Hz, 5H), 3.42 (t, J=6.4 Hz, 1H), 2.34 (dd, J=5.6, 2.9 Hz, 1H), 2.21-1.96 (m, 5H), 1.86-1.73 (m, 5H), 1.57-1.49 (m, 1H), 1.39 (d, J=10.2 Hz, 1H), 1.25-1.20 (m, 6H), 0.98 (dd, J=10.6, 6.7 Hz, 6H), 0.93-0.86 (m, 6H), 0.81 (d, J=3.2 Hz, 3H).

MS [M+H]⁺=667

Step 6: Lithium hydroxide monohydrate (0.42 g) was weighed, added and dissolved to 9.0 g of purified water (9.0 g) for later use. Under nitrogen protection, the lithium hydroxide aqueous solution was dropwise added to the reactor that contains compound 9 (5.0 g) in tetrahydrofuran, at −10° C. After that, the solution was heated to 5° C. and stirred for 10 h. After the reaction was completed, added purified water (9.0 g) to the reaction solution at 5° C., and then extracted by MTBE (16.6 g) for twice. The aqueous phase and the organic phase were separated, and the organic phase in two extractions were combined to give the organic phase 6-A which was temporarily stored. After the addition of isopropyl acetate (16.6 g) for extraction twice at 5° C., the aqueous phase was slowly added with 1M hydrochloric acid solution (11.42 g) for adjusting the pH to 2-3 and placed for separation. The organic phase obtained in this two-time extraction were collected and combined to give the organic phase 6-B. After adding purified water (15.0 g) to the combination of the organic phase 6-A and 6-B for extraction, the collected organic phase 6-C was concentrated to 5-10 mL under reduced pressure. Tetrahydrofuran (19.7 g) was added and dissolved to the concentrated residues which were then concentrated, and this operation was repeated twice. Then, after supplementary addition and dissolution of tetrahydrofuran (15.0 g) to the concentrated residues, the reactor was added with n-heptane (100.0 g), and the organic solvent (THF solution, the final weight ratio of the crystallization solvent THF to n-heptane is 1:5) dropwise. After that, the solution was stirred for 1 h and filtered, and the filter cake was washed with heptane (10.0 g), and dried to give a white solid (4 g) having a purity of 98.7% and a yield of 82%.

The characterization data of compound 10:

¹H NMR (400 MHZ, DMSO-d6) δ 12.66 (s, 1H), 8.97 (t, J=5.9 Hz, 1H), 8.87 (t, J=3.5 Hz, 1H), 8.69 (d, J=8.1 Hz, 1H), 8.02-7.90 (m, 4H), 4.57-4.44 (m, 2H), 4.07 (dd, J=8.7, 2.2 Hz, 1H), 3.95 (d, J=5.8 Hz, 2H), 3.91-3.85 (m, 1H), 3.69 (ddd, J=9.5, 7.2, 5.3 Hz, 1H), 2.34 (dd, J=5.5, 2.9 Hz, 1H), 2.25-2.07 (m, 3H), 2.03-1.89 (m, 3H), 1.86-1.72 (m, 4H), 1.65 (dt, J=13.8, 2.8 Hz, 1H), 1.39 (d, J=10.0 Hz, 1H), 1.22 (d, J=1.1 Hz, 6H), 1.11 (s, 5H), 0.98 (dd, J=10.2, 6.7 Hz, 6H), 0.90 (dd, J=13.8, 6.8 Hz, 6H), 0.81 (s, 3H).

MS [M+H]⁺=653

Step 7: Under the protection of nitrogen, acetonitrile (12.0 g), TFA (0.87 g), isobutylboronic acid (2.35 g) and compound 10 (5.0 g) were successively added into the reactor. After three times of nitrogen replacement, the reaction solution was stirred at 40° C. for 18-24 h. After the reaction was completed, the reaction solution was added with water (2.0 g) under stirring and then n-heptane for extraction twice (15.0 g of n-heptane was added each time). The bottom organic phase from each extraction was collected, combined and placed in a reactor that was then added with D301 basic resin (2.5 g) and stirred for 1-2 h. After removal of the resin by filtration at 30° C. and collection of the organic phase, the filtered D301 resin was washed by a mixed solution (7.5 mL) of acetonitrile and water (volume ratio of acetonitrile:water=9:1). The combination of the organic phase and the washing liquid was filtered by a precision filter and the filtrate was collected and added dropwise to the antisolvent MTBE (300 g). After that, this solution was stirred for 0.5-1 h, cooled down to 5° C., kept stirred for 0.5-1 h, and filtrated. The filter cake was washed with MTBE (10 g) to give a white solid (3 g) having a purity of 97.3% and a yield of 75%.

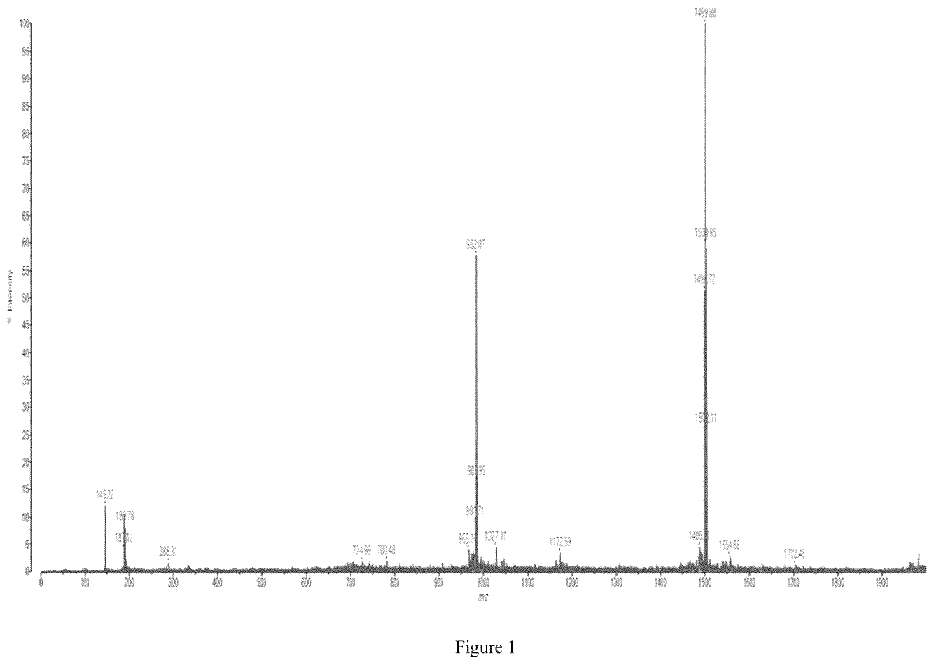

The elemental analysis results of the compound of Formula (I) were shown in Table 1, and the mass spectrum of the compound of Formula (I) was shown in FIG. 1.

TABLE 1

| | | | Result after | Absolute |
|---|---|---|---|---|
| | Theoretical | Measured | Adjusting for | Deviation |
| Element | Value (%) | Value (%) | Residue (%) | (%) |
| C | 57.61 | 57.09 | 57.16 | 0.45 |
| H | 6.65 | 6.63 | 6.64 | 0.01 |
| N | 11.20 | 11.32 | 11.33 | 0.13 |
| B | 2.16 | 2.18 | 2.18 | 0.02 |
| O | 22.38 | / | 22.69 | 0.31 |

Note
The residue result is 0.13%

¹H-NMR (400 MHZ, D₂O) δ=7.82-7.89 (0, 4H), 4.60 (d, J=8.5 Hz, 1H), 4.55 (dd, J=6.5, 2.0 Hz, 1H), 4.18 (s, 2H), 3.96-4.08 (m, 1H), 3.75-3.87 (m, 1H), 2.40-2.52 (m, 1H), 2.27-2.36 (m, 1H), 2.12-2.23 (0, 2H), 1.97-2.09 (0, 2H), 1.66-1.82 (m, 1H), 0.98-1.12 (m, 6H), 0.84-0.96 (m, 6H).

MS-negative ion: 1499.88 [M−H]⁻

### Example 2 Activity Test of Compound (I) Against Recombinant Human Elastase (rhElastase)

Activation of protease: rhElastase was diluted to 2 μM with an activation buffer containing 1.25 μM Recombinant Mouse Active Cathepsin C Protein (mCathepsin C), and incubated at 37° C. for 2 h. After activation, the mature rhElastase was diluted to form a 3.33 nM rhElastase reaction solution. The compound was diluted in serial with DMSO and added to a 384-well plate (3-fold serial dilution, 10 concentration points, duplicate wells). For the 384-well plate containing the compound, columns 1-12 each was added with 15 μL of the reaction buffer and columns 13-24 each was added with 15 μL of the rhElastase reaction solution. The plate was pre-incubated at 25° C. for 30 min and added with 5 μL of the substrate to start the reaction. The final concentration of rhElastase is 2.5 nM, and the final concentration of the substrate is 3.125 μM. The final concentration of DMSO in the reaction system is 1%. After incubating at 37° C. for 60 min, the fluorescence value was read with an M4 microplate reader. The detection conditions are Ex/Em=380 nm/460 nm.

Result: The result shows that the IC₅₀ value of compound (I) against rhElastase is 1.318 nM.

### Example 3: Pharmacokinetic Experiment of Compound (I)

The appropriate amount of sodium citrate was weighed and dissolved in water to prepare a sodium citrate solution with a concentration of 1 mg/ml. After the addition of compound (I) (appropriate amount) under stirring, the sodium citrate solution was slowly added with 1 M sodium hydroxide solution to adjust the pH to 5.0±0.2. After stirring, dissolving and filtering, a stock solution of compound (I) with a concentration of 80 mg/ml was obtained, which is administrated by inhalation. The stock solution was diluted in a continuous gradient method to give a solution of compound (I) with a concentration of 0.4 mg/ml for intravenous administration.

21 male Sprague-Dawley rats was selected and divided into 7 groups (3 animals/group). Group 1 was administered intravenously at a dose of 2 mg/kg, and groups 2-7 are administered by inhalation at a designed dose of 15 mg/kg (an actual delivered dose is 16.712 mg/kg). For animals in groups 1-2, blood samples were collected before administration and at 2 min, 5 min, 15 min, 30 min, 1 h, 2 h, 4 h, 6 h, 8 h, and 24 h after administration. For animals in groups 3-7, lung tissues and blood samples were collected at 0.25 h, 1 h, 2 h, 6 h, and 12 h after administration (i.e., 2.25 h, 3 h, 4 h, 8 h, and 14 h After the start of the drug administration). The LC-MS/MS was used to analyze the concentration of compound (I) in the plasma and lung tissues of rats, and the non-compartmental model (NCA) of the pharmacokinetic analysis software WinNonlin (8.0.0.3176) is used to analyze the drug concentrations in plasma and lung tissue and calculate the pharmacokinetic parameters.

The main pharmacokinetic parameters of compound (I) in Sprague-Dawley rats by intravenous/gavage/inhalation administration are shown in Table 2.

TABLE 2

| Administration | Dose | t_{1/2} | Tₘₐₓ | Cₘₐₓ | AUCₗₐₛₜ | |
|---|---|---|---|---|---|---|
| form | (mg/kg) | (h) | (h) | ng/mL | h*ng/mL | F % |
| Intravenous | 2 | 0.33 ± 0.10 | 0.03 ± 0.0 | 5331.49 ± 696.49 | 1107.18 ± 144.07 | / |
| Inhalation | 15 | 1.91 ± 1.05 | 2.03 ± 0.0 | 798.55 ± 333.20 | 1501.79 ± 486.138 | 18.09 |

After administered by inhalation to Sprague-Dawley rats, the exposure amounts of the compound (I) in the lungs and plasma of the rats are shown in Table 3.

TABLE 3

| Dose | | t_{1/2} | Tmax | | |
|---|---|---|---|---|---|
| (mg/kg) | Matrix | (h) | (h) | Cmax | AUClast |
| 15 | Lung | 2.69 | 2.25 | 67568.60 | 149415.60 |
| | Plasma | — | 2.25 | 320.58 | 622.35 |

### Example 4: Preparation of Crystal Form a of the Compound Shown in Formula (I)

Crystal form A can be obtained by room temperature suspension stirring in an ACN/H₂O (88:12, v/v) solution.

Method 1: Approximately 20 mg of the starting sample was weighed in an HPLC vial, added with 0.5 mL of the solvent ACN/H₂O (94:6, aw˜0.73), and magnetically stirred at room temperature to give a suspension. The solid was obtained by separating the suspension after about 7 days, which is a crystal form A characterized by XRPD.

Method 2: Approximately 20 mg of the starting sample was weighed in an HPLC vial, added with 0.5 mL of the solvent ACN/H₂O (89:11, aw˜0.89), and magnetically stirred at room temperature to give a suspension. The solid was obtained by separating the suspension after about 7 days, which is a crystal form A characterized by XRPD.

Method 3: Approximately 20 mg of the starting sample was weighed in an HPLC vial, added with 0.5 mL of the solvent ACN/H₂O (81:19, aw˜0.93), and magnetically stirred at room temperature to give a suspension. The solid was obtained by separating the suspension after about 7 days, which is a crystal form A characterized by XRPD.

Method 4: Approximately 20 mg of the starting sample was weighed in an HPLC vial, added with 0.5 mL of water, and magnetically stirred at room temperature to give a suspension.

The suspension becomes clear after 7 days and continues to stir after adding solids. Then, the solid was obtained by separating the suspension which is a crystal form A characterized by XRPD.

The XRPD spectrum is shown in FIG. 2, and the characteristic peak positions are shown in Table 4.

TABLE 4
XRPD Diffraction Peaks of Crystal Form
A for the Compound of Formula (I)

| Diffraction peak position [°2Theta] | Relative peak intensity [%] |
|---|---|
| 8.48 | 12.03 |
| 9.18 | 100.00 |
| 10.32 | 13.38 |
| 11.46 | 12.92 |
| 11.70 | 9.54 |
| 13.09 | 21.95 |
| 13.52 | 14.85 |
| 15.76 | 14.28 |
| 16.14 | 17.50 |
| 16.73 | 20.14 |
| 17.08 | 23.56 |
| 18.47 | 32.75 |
| 19.72 | 6.42 |
| 20.04 | 10.42 |
| 21.09 | 16.23 |
| 21.62 | 20.14 |
| 22.29 | 11.50 |
| 22.87 | 15.86 |
| 24.32 | 5.66 |
| 25.99 | 13.31 |
| 26.43 | 5.76 |

### Example 5: Preparation of Crystal Form A of the Compound Shown in Formula (I)

200.15 mg of the compound of Formula (I) was weighed into a 4-ml vial, added with 1 ml of ACN/H₂O (88:12, v/v), then added with seeds of crystal form A, and stirred for about two days at room temperature. The wet sample contains crystal form A characterized by XRPD. Then wet sample was filtered, and the filter cake was dried at room temperature to give the solid. The XRPD results show that the solid sample prepared by repeated sample preparation is a highly crystalline crystal form A.

### Example 6: Preparation of the Amorphous Form of the Compound Shown in Formula (I)

100 mg of the compound of Formula (I) was weighed into a 4-ml vial and added with 0.5 ml of ACN/H₂O (4:1, v/v), which were heated to 50° C. and stirred for dissolution. The solution was cooled down to 5° C., added to 2.5 ml acetonitrile dropwise, stirred at 5° C. for 4 h, filtered, and dried in vacuum to give the amorphous product. The XRPD spectrum of the amorphous product is shown in FIG. 4.

### Example 7: Mechanical Stability of Crystal Form A of the Compound Shown in Formula (I)

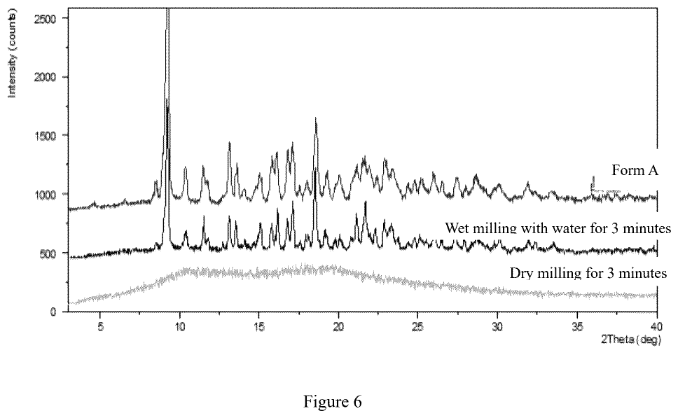

A certain amount of crystal form A of the compound shown in Formula (I) was weighed, and ground dry or wet with an appropriate amount of water for 3 minutes at room temperature (˜23° C., 70% RH) to evaluate the mechanical stability of the crystal form A. The XRPD spectrums are shown in FIG. 6. The results show that the crystal form A is transformed into the amorphous form after dry grinding, and remains unchanged after wet grinding with water. The specific results are shown in Table 5:

TABLE 5
Results of the grinding experiment using crystal form A

| | Start Crystal | | Achieved Crystal |
|---|---|---|---|
| No: | Form | Method | Form |
| 1 | Form A | Dry grind for 3 minutes | Amorphous |
| 2 | Form A | Wet grind with water for 3 | Form A |
| | | minutes | |

### Example 8: Solid-State Stability for Crystal Form A and Amorphous Form of the Compound of Formula (I)

A certain amount of crystal form A and amorphous form of the compound shown in Formula (I) were respectively taken, and the stabilities of the crystal form A and amorphous form were evaluated under different stability conditions (25° C./60% RH, 40° C./75% RH, 60° C.). The experimental results are shown in Table 6.

TABLE 6
2-Week Stability Study of Crystal Form A and Amorphous Form

| | | 1 week | | | | 2 weeks | | | |
|---|---|---|---|---|---|---|---|---|---|
| | | | Relative | | | | Relative | | |
| Crystal | | Purity | to initial | Content | Crystal | Purity | to initial | Content | Crystal |
| form | Conditions | (area %) | (%) | (%) | Form | (area %) | (%) | (%) | form |
| Amorphous | initial | 98.56 | — | 90.28 | Amorphous | 98.56 | — | 90.28 | Amorphous |
| | 25° C./ | 97.58 | 99.0 | 88.54 | Amorphous | 96.98 | 98.4 | 86.62 | Amorphous |
| | 60 % RH | | | | | | | | |
| | 40° C./ | 97.87 | 99.3 | 87.54 | Amorphous | 96.87 | 98.3 | 86.77 | Amorphous |
| | 75 % RH | | | | | | | | |
| | 60° C. | 96.97 | 98.4 | 90.96 | Amorphous | 96.14 | 97.5 | 88.93 | Amorphous |
| Form A | initial | 99.05 | — | 87.08 | Form A | 99.05 | — | 87.08 | Form A |
| | 25° C./ | 97.98 | 98.9 | 86.07 | Form A | 97.68 | 98.6 | 86.60 | Form A |
| | 60 % RH | | | | | | | | |
| | 40° C./ | 97.96 | 98.9 | 86.41 | Form A | 97.76 | 98.7 | 86.85 | Form A |
| | 75 % RH | | | | | | | | |
| | 60° C. | 98.16 | 99.1 | 87.34 | Form A | 97.50 | 98.4 | 87.98 | Form A |

Note:
The content was estimated by using an amorphous sample as the reference standard. The content of the reference standard was calculated based on the weight loss in thermogravimetric analysis (TGA) (8.68%) and the high-performance liquid chromatography (HPLC) purity (98.56%) as follows: assay = purity * (1 − TGA loss) = 90.00%.

From the above, it can be concluded that neither the amorphous form nor crystal form A underwent crystal form changes in the two-week stability assessment.

A certain amount of crystal form A and amorphous form of the compound shown in Formula (I) were respectively taken, and the stabilities of crystal form A and amorphous form were evaluated under light conditions. The experimental results are shown in Table 7.

TABLE 7
Photostability of Crystal Form A and Amorphous Form

| | | | | After 11 days | | | |
|---|---|---|---|---|---|---|---|
| Initial Sample | | | | | Relative | | |
| Crystal | Purity | Content | | Purity | to initial | Content | Crystal |
| form | (area %) | (%) | Conditions | (area %) | (%) | (%) | form |
| Amorphous | 98.56 | 90.28 | Temperature: | 93.42 | 94.8 | 82.68 | Amorphous |
| | | | 25° C., | | | | |
| | | | Illumination | | | | |
| | | | intensity: | | | | |
| | | | 5000 lux/h, | | | | |
| | | | Near-ultraviolet | | | | |
| | | | intensity: | | | | |
| | | | 90 μW/cm² | | | | |
| Form A | 99.05 | 87.08 | Temperature: | 97.92 | 98.9 | 85.00 | Form A |
| | | | 25° C., | | | | |
| | | | Illumination | | | | |
| | | | intensity: | | | | |
| | | | 5000 lux/h, | | | | |
| | | | Near-ultraviolet | | | | |
| | | | intensity: | | | | |
| | | | 90 μW/cm² | | | | |

Note:
The content was estimated by using an amorphous sample as the reference standard. The content of the reference standard was calculated based on the weight loss in thermogravimetric analysis (TGA) (8.68%) and the high-performance liquid chromatography (HPLC) purity (98.56%) as follows: assay = purity * (1 − TGA loss) = 90.00%.

The results show that neither the amorphous form nor crystal form A underwent crystal form changes in the photostability test.

### Example 9: Dynamic Solubility of Crystal Form A and Amorphous Form of the Compound Shown in Formula (I)

A certain amount of crystal form A and amorphous form of the compound shown in Formula (I) were taken and dissolved in citrate buffer solutions (pH=3.5, 4.5) at room temperature for testing the dynamic solubility. The preparation of citrate buffer solutions was shown in Table 8.

TABLE 8
Preparation of Citrate Buffer Solutions (pH = 3.5, 4.5)

| | Mass (mg) | | | |
|---|---|---|---|---|
| | | Dihydrate | | |
| | Anhydrous | trisodium | | |
| pH | citric acid | citrate | Volume | Preparation method |
| 3.5 | 3386.88 | 2168.46 | 50 ml | Step 1: Corresponding mass of |
| 4.5 | 2257.54 | 3897.23 | 50 ml | anhydrous citric acid or |
| | | | | dihydrate trisodium citrate was |
| | | | | weighed and dissolved in |
| | | | | approximately 40 mL of purified |
| | | | | water. |
| | | | | Step 2: The buffer solution was |
| | | | | obtained by adjusting the |
| | | | | corresponding pH values with |
| | | | | 1N NaOH or 1N HCl, and |
| | | | | supplying pure water at room |
| | | | | temperature to make up the |
| | | | | volume to 50 mL. |

During the test, about 200 mg of crystal form A or the amorphous form was added into 5 mL of buffered salt medium (approximately 40 mg/mL), and stirred magnetically at room temperature (approximately 25° C.). 1 mL of the suspension (or clear solution) taken at each of 2, 8, and 30 hours were centrifugated. The obtained solid is for crystal form detection (XRPD), and the supernatant/clear liquid after filtration is for solubility testing (HPLC) and pH testing. The results are shown in Table 9.

TABLE 9
Solubility of the Amorphous Form and Crystal Form A at Room Temperature
Solubility in Citrate Buffer Solution at Room Temperature (pH = 3.5)

| | 2 hours | | | 8 hours | | | 30 hours | | |
|---|---|---|---|---|---|---|---|---|---|
| | | | Crystal | | | Crystal | | | Crystal |
| | Solubility | | form | Solubility | | form | Solubility | | form |
| Sample | (mg/ml) | pH | change | (mg/ml) | pH | change | (mg/ml) | pH | change |
| Amorphous | >37.77 | 3.63 | Dissolved | >38.95 | 3.59 | Dissolved | >37.45 | 3.56 | Dissolved |
| | | | clarification | | | clarification | | | clarification |
| form A | 6.15 | 3.56 | Crystal | 15.28 | 3.58 | Crystal | 27.16 | 3.56 | Crystal |
| | | | form A | | | form A | | | form A |

Solubility in Citrate Buffer Solution at Room Temperature (pH = 4.5)

| | | | | | | | | | |
|---|---|---|---|---|---|---|---|---|---|
| Amorphous | >33.47 | 4.46 | Dissolved | >35.71 | 4.44 | Dissolved | >37.52 | 4.40 | Dissolved |
| | | | clarification | | | clarification | | | clarification |
| form A | 16.06 | 4.48 | Crystal | 31.34 | 4.46 | Crystal | >34.94 | 4.38 | Crystal |
| | | | form A | | | form A | | | form A |

The results show that the amorphous form exhibits excellent solubility in the citrate buffer systems with pH=3.5 and pH=4.5

It should be understood that the above examples are all exemplary and are not intended to cover all possible embodiments encompassed by the claims. Various modifications and changes can still be made on the basis of the above examples without departing from the scope of the present disclosure. Similarly, the various technical features of the above examples can also be arbitrarily combined to form additional examples that may not have been explicitly described herein. Therefore, the above examples only express several embodiments of the present invention and do not limit the protection scope of the present invention.

1. A compound of formula I, or a pharmaceutically acceptable salt, ester, isomer, solvate, prodrug or isotopically labeled derivative thereof,

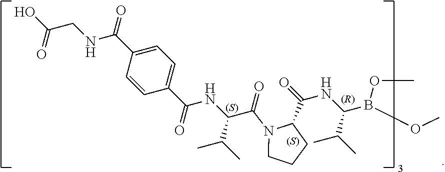

2. A crystal form A of the compound of Formula (I) according to claim 1, having 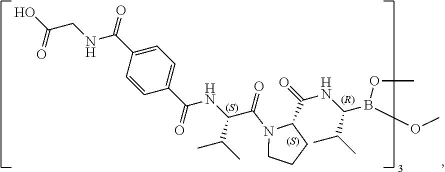
an X-ray powder diffraction pattern comprising, in terms of 2θ angle, peaks at 8.48, 9.18, 13.09, 17.08 and 18.47.

3. The crystal form A according to claim 2, wherein the 2θ angle has an error range of ±0.20.

4. An amorphous form of the compound of Formula (I) according to claim 1,

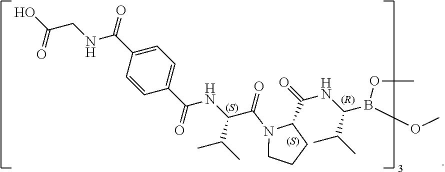

5. The amorphous form according to claim 4, having an X-ray powder diffraction pattern substantially as shown in FIG. 4, in terms of 2θ angle.

6. A preparation method of the crystal form A according to claim 2, comprising:
using the amorphous form of the compound of Formula (I) as a raw material and preparing the crystal form A by a method selected from gas-solid diffusion, gas-liquid diffusion, slow evaporation, suspension stirring at room temperature to 50° C., temperature cycle stirring, slow-cooling and polymer induction.

7. The preparation method of the crystal form A according to claim 2, comprising mixing the compound of Formula (I) with an aqueous solution of solvent 1, and then stirring and filtering;
wherein the solvent 1 is selected from nitrile solvents; and
wherein a volume ratio of solvent 1 to water is (5-10):1.

8. A preparation method of the amorphous form of the compound of Formula (I) according to claim 4, comprising: adding solvent 3 to a solution 2 of the compound of Formula (I), cooling down to 0-10° C., stirring, and filtering;
wherein a solvent of the solution 2 is one or more selected from 1,4-dioxane, tetrahydrofuran, 2-methyltetrahydrofuran, ethylene glycol dimethyl ether, dichloromethane, dimethyl sulfoxide, acetone, acetonitrile and water;
wherein a volume ratio of acetonitrile to water is (4-9):1;
wherein the solvent 3 is selected from methyl tert-butyl ether, acetonitrile, isopropyl acetate; and
wherein a volume ratio of the solution 2 to the solvent 3 is 1:(5-15), preferably 1:(5-10).

9. A pharmaceutical composition prepared from the compound of Formula (I) according to claim 1.

10. A pharmaceutical composition comprising the compound of Formula (I) according to claim 1 or a pharmaceutically acceptable salt, ester, isomer, solvate, prodrug or isotopically labeled derivative thereof.

11. A preparation method of a pharmaceutical composition, comprising mixing compound of Formula (I) according to claim 1 or a pharmaceutically acceptable salt, ester, isomer, solvate, prodrug or isotopically labeled derivative thereof.

12. The compound of Formula (I) according to claim 1 or a pharmaceutically acceptable salt, ester, isomer, solvate, prodrug or isotopically labeled derivative thereof.

13. The use according to claim 12, wherein the diseases mediated by elastase is selected from chronic obstructive pulmonary disease, bronchiectasis, cystic fibrosis, acute lung injury, chronic bronchitis, acute respiratory distress syndrome, pulmonary artery hypertension, idiopathic pulmonary fibrosis and alpha-1 antitrypsin deficiency, preferably is chronic obstructive pulmonary disease.

14. The crystal form A of the compound of Formula (I) according to claim 2, wherein the X-ray powder diffraction pattern comprises, in terms of 2θ angle, peaks at 8.48, 9.18, 10.32, 11.46, 13.09, 16.14, 16.73, 17.08, 18.47, 21.09 and 21.62.

15. The crystal form A of the compound of Formula (I) according to claim 2, wherein the X-ray powder diffraction pattern comprises, in terms of 2θ angle, peaks at 8.48, 9.18, 10.32, 11.46, 11.70, 13.09, 13.52, 15.76, 16.14, 16.73, 17.08, 18.47, 21.09, 21.62, 22.87 and 25.99.

16. The crystal form A of the compound of Formula (I) according to claim 2, wherein the X-ray powder diffraction pattern comprises, in terms of 2θ angle, a X-ray powder diffraction pattern substantially as shown in FIG. 2.
